(19) United States
(12) Patent Application Publication
(10) Pub. No.: US 2004/0068226 A1
(43) Pub. Date: Apr. 8, 2004

(54) Osteodwelling catheter

(76) Inventor: James Kevin Brannon, Culver City, CA (US)

Correspondence Address: James K. Brannon, M.D., 5729 Canterbury Drive, Culver City, CA, 90230, US

(21) Appl. No.: 10/263,035

(22) Filed: Oct. 2, 2002

(51) Int. Cl. A61M 29/00

(52) U.S. Cl. 604/101.01

(57) ABSTRACT

An osteodwelling catheter is described for partitioning an osteonecrotic segment of bone in patients with osteonecrosis. The osteodwelling catheter includes a first inflatable balloon designed to induce a collection of platelets to aggregate and degranulate so as to induce hemostasis thereabout the inflatable balloon. A second inflatable balloon is provided so as to induce a collection of platelets to aggregate and degranulate and thereby induce hemostasis locally thereabout the inflated balloon. The partitioning realized by the inflatable balloons allows one to deliver a contrast material to the partitioned segment of bone or alternately measure an arterial or venous pressure. The osteodwelling catheter is passed through an endoscope so as to allow one to observe directly where the balloons are to be inflated inside of the femoral neck. Several embodiments of the osteodwelling catheter are described.

### BACKGROUND OF THE INVENTION

1. Field of the Invention

Osteonecrosis of the femoral head in the young patient is a musculoskeletal disorder with growing concerns, particularly as osteolysis from particulate polyethylene wear debris compromises the longevity of a total hip arthroplasty. Approximately 20,000 new cases are reported each year, with an estimated 450,000 patients, on average, with ongoing disease in the United States. Lavernia et al. further reported in the Journal of the American Academy of Orthopaedic Surgeons in 1999 that osteonecrosis usually occurs during the prime of one's working years.

Osteonecrosis of the femoral head can be separated into two clinical categories, the symptomatic hip and the asymptomatic hip. Almost uniformly, 85% of symptomatic hips progress to collapse, irrespective of the stage of disease at the time of the initial diagnosis. It is often the asymptomatic hip wherein controversy arises regarding treatment. Urbaniak found in his series of asymptomatic patients that at least ⅔ would progress to collapse. Importantly, one may define impending collapse of the femoral head as greater than 50% head involvement in two radiographic orthogonal views. Bradway and Morrey, in the J of Arthroplasty 1993, found that a collection of 15 “presymptomatic” hips all collapsed. Consequently, proponents of core decompression recommend early diagnosis and treatment of disease, with the understanding that such a treatment regimen may not halt progression.

Many theories have been proposed to explain the pathogenesis of osteonecrosis of the femoral head, as the name itself seems to describe the end condition, dead or nonviable osteocytes surrounded by a matrix of mineralized bone. More importantly, at least five categories have been identified as a potential mechanisms underlining the basis for disease: (1) Direct Cellular Mechanisms, cells die as a result of chemotherapy or thermal injury; (2) Extraosseous Arterial Mechanisms, ischemic necrosis of the femoral head following a substantially displaced fracture of the femoral neck; (3) Extraosseous Venous Mechanisms, an observation supported by the work of Ficat and Arlet in which these investigators observed venous hypertension in all clinical stages of osteonecrosis. Interestingly, The Johns Hopkins University observed compensatory mechanisms in the venous outflow of the femoral head when the venous system was obstructed using a dog model, raising questions about the role of venous congestion in the pathogenesis of disease; (4) Intraosseous Extravascular Mechanisms, this finding is thought to be consistent with bone marrow edema often observed on magnetic resonance imaging; and (5) Intraosseous Intravascular Mechanisms, occlusion of small vessels in patients with sickle-cell disease and dysbaric exposure wherein emboli of fat or nitrogen bubbles are thought to lead to osteonecrosis of the femoral head.

At least four Stages of osteonecrosis are described to allow one to institute and compare various treatment regimens. The most frequently used staging system is that of Ficat and Arlet as follows: Stage I, normal plain film radiographs; Stage II, Sclerotic or cystic lesions without subchondral fracture; Stage III, Subchondral fracture,(crescent sign), with or without articular incongruity; and Stage IV, osteoarthrosis with osteophytes. Other staging systems include those of Marcus et al., University of Pennsylvania System of Staging, Association Research Circulation Osseous (ARCO), and the Japanese Investigation Committee on Osteonecrosis wherein the location of the lesion determines the stage of disease.

At the histologic level, necrosis of the femoral head can be described as dead or nonviable osteocytes surrounded by a mineralized matrix of bone. In a retrospective study by Marcus and Enneking, 13 core biopsies had been performed to treat eleven patients with asymptomatic or silent hips in Stage I or Stage II disease. All core biopsies in their series demonstrated normal articular cartilage, necrotic subchondral bone, and creeping substitution (osteoclastic bone resorption followed by the infiltration of marrow mesenchymal cells within a fibrovascular stroma). These observations and those of Phemister, Bonfiglio and others suggest that the success of core decompression in the treatment of osteonecrosis of the femoral head, Stage I or II, partly depends on the ability of autologous bone graft to incorporate the necrotic segment of bone within the femoral head. However, these authors did not attempt to characterize the requirements for host bone incorporation beyond ant adequate blood supply.

The diagnosis of osteonecrosis can be easily made on plain film radiographs, assuming the disease is at least Ficat and Arlet Stage II, combined with a thorough history with an emphasis on predisposing risk factors, principally alcohol and steroid use, and a complete physical examination. Magnetic resonance imaging (MRI) may add additional information but is not routinely necessary. The MRI, however, is particularly useful in the asymptomatic hip, Ficat and Arlet Stage I.

Treatment options for osteonecrosis of the femoral head are categorized into one of two major groups, non-operative and operative. Nonoperatively, limited clinical success has been observed in the treatment of the symptomatic hip. Mont and Hungerford reviewed the nonoperative experience in the medical literature and found that only 22% of 819 hips in several pooled studies had a satisfactory result. These authors refer to the location of the osteonecrotic lesion, medial versus lateral, and suggest that medial lesions are more likely to have a satisfactory outcome. This observation is consistent with a mechanical component having a dominant role in the progression of disease, irrespective of etiology. Alternatively, operative treatment can be characterized as core decompression of the femoral head with or without bone grafting followed by at least six weeks of non-weight bearing. Brown et al. at the University of Iowa used a three-dimensional finite-element model to elucidate the stress distribution over the diseased femoral head so as to characterize the optimal placement of a decompressing core with respect to location, depth, and diameter. More importantly, Brown et al. further showed that the optimum mechanical benefit of appropriately placed cortical bone grafts in a decompressed femoral head is realized when such grafts are situated in direct mechanical contact with the subchondral plate. These authors used the gait cycle to identify peak stress in the femoral head during normal walking and concluded that when fibula grafts are appropriately placed they potentially afford relief of stress to vulnerable necrotic cancellous bone in the subchondral and superocentral regions of the femoral head, implying that osseous incorporation of the cortical bone graft may be ideal but not completely necessary in the prevention of collapse. Although Brown et al. outlined the importance of strategic placement of a cortical fibula graft, it is important to recognize that these authors assumed that the necrotic cancellous bone is at risk for an intra-substance fracture, in the absence of treatment and that such intra-substance structural failure is principally responsible for the progression of disease, i.e., collapse of the femoral head. One must consider that as a segment of the femoral head becomes increasingly necrotic, its modulus of elasticity may vary substantially from that of the surrounding cancellous bone, and that progression of disease is perhaps also failure of the surrounding bone at the necrotic host bone interface; the area of creeping substitution in the work of Bonfiglio et al. Although not a part of the investigative objective, Brown et al. additionally did not demonstrate how cyclic loading of a cortical bone graft beneath the subchondral plate influences the healing behavior of the surrounding necrotic bone at the host necrotic bone interface. More specifically, is bony union achieved at the necrotic host bone interface now that the necrotic bone is unloaded? Is the fibula strut really a load-bearing cortical graft to the extent that the surrounding necrotic bone no longer sustains a substantial cyclic load during gait? Does the fibula strut simply allow the joint reactive force to bypass the segment of necrotic bone thereby substantially reducing its micromotion? Does micromotion of the necrotic segment of bone cause pain? Does the pain spectrum associated with osteonecrosis suggest a nonunion at the necrotic host bone interface, an intraosseous nonunion? These questions and others are prompted by the observation of good to excellent outcomes in patients with Ficat and Arlet Stage I or Stage II disease treated with core decompression with vascular or avascular bone grafts, keeping aside the retrospective results of Kim et al. presented at the 1998 Annual Meeting comparing vascular to avascular fibula struts in treating osteonecrosis. More importantly, patients have been shown to benefit from core decompression alone implying that increased intraosseous pressure may play a dominant role in the early stages of disease, whereas in the later stages, the necrotic bone is less ductile and behaves in a brittle fashion giving rise to subchondral collapse as evidence for a mechanical component playing a dominant role in the progression of later stage disease. Recently, Mont et al. reported in the Journal of Bone and Joint Surgery good to excellent results in two groups of six dogs, twelve osteonecrotic hips, treated with trans-articular decompression of the femoral head and bone grafting, with and without osteogenic protein-1. Although the authors sought to elucidate the difference in healing time, i.e., the time to graft incorporation between the two groups, the critical observation is that all twelve hips were treated with avascular autograft. Therefore, Mont's work in view of Brown et al. causes one to consider the role a vascularized fibula graft in the treatment of osteonecrosis of the femoral head. Does revascularization really occur secondary to the blood supply thought to be provided by a vascularized fibula graft?

The work of Brown et al. suggests that core decompression is substantially core débridement of the femoral head. However, as one attempts to adequately débride the femoral head of osteonecrotic bone, the diameter of the core, by necessity, becomes increasingly large because one is not able to mechanically débride bone from the femoral head at a right angle to the central axis of the decompressing core. Furthermore, strategic placement of a cortical bone graft beneath the subchondral plate simply provides means for bypassing the at risk necrotic bone and transfers the load during gait to the fibula strut. The addition of a “blood supply,” vascularized fibula graft, in part relies on the work of Bonfiglio et al. Interestingly, “necrotic” autogenous bone stimulates osteoclastic bone resorption. In a recent issue of the Journal of Bone and Joint Surgery, Enneking showed in a histopathologic study that massive preserved human allografts (avascular bone) are slowly incorporated into host bone through limited bridging external callus and internal repair, even when rigid fixation is used to stabilize these grafts. Enneking suggests that the limited incorporation of allograft at cortical-cortical junctions could be enhanced with more recently developed osteoinductive substances. Importantly, however, is that Enneking observed enhanced bridging callus formation that lacked remodeling along the lines of stress, at allograft host junctions that were augmented with autogenous bone. Enneking did not observe increased internal repair that characterizes graft incorporation. The critical issues is that bone resorption must be followed by the infiltration of mesenchymal cells within a fibrovascular stroma for true incorporation to be established. Viable autograft appears to retain its ability to induce ongoing osteoclastic resorption and new bone formation, whereas allograft lacks this ability, as it is principally osteoconductive. A blood supply may be more important at cortical-cortical junctions. Cortical-cancellous junctions depend on the nature of the host cancellous bone. Cortical bone will not incorporate necrotic cancellous bone as cortical bone lacks sufficient metabolic activity. However, given that cancellous bone substantially more porous than cortical bone and is further 8 times as metabolically active as cortical bone, one can expect incorporation of viable cortical bone at a cortical-cancellous junction. Consider the following: Within the growth plate, necrotic calcified cartilage stimulates osteoclastic resorption followed by the laying down of osteoid by osteoblast. In primary bone healing, osteoclast bore into necrotic segments of bone, which are then followed by the laying down of osteoid by osteoblast. One might recognize that in these examples, necrotic calcified cartilageand avascular autogenous bone both induce the infiltration of osteoclast and mesenchymal cells. Further, external bridging callus in the presence of internal repair represents union. Enneking's work suggest that external bridging callus along human allograft bone is a surface event driven by local mesenchymal cells in the surrounding tissue, whereas internal repair is limited as the cytokines germane to new bone formation within the substance of the allograft bone are compromised during the sterilization process.

Einhorn et al. have shown that despite the great ingrowth of capillaries into fracture callus, cell proliferation is such that the cells exist in a state of hypoxia. This hypoxic state could be favorable for bone formation, as in-vitro bone growth optimally occurs in a low-oxygen environment. Therefore, avascular autogenous bone, in and of itself, is not “bad” bone. Necrotic bone (a necrotic segment of bone within the femoral head) retains its osteoinductivity and osteoconductivity. Osteoinduction is an avascular physiologic event dependent on BMP's within the substance of the bone in question and the blood supply of the surrounding tissue being induced, whereas osteoconduction is an avascular, physical event dependent on the structural integrity of the inorganic extracellular matrix of bone. Urist in the Journal of Science in 1965 showed that “avascular” demineralized bone implanted in extra-skeletal sites would induce bone formation. Enneking has shown recently in the Journal that new bone formation (bridging callus) can occur with massive allografts (necrotic bone) but internal repair (an avascular physiologic event) is limited. Importantly, human allograft bone lacks osteoinduction sufficient to promote internal repair characteristic of bony union, as allograft bone is “processed” bone and consequentially, it may lose its ability to induce new bone formation. Necrotic or avascular autogenous bone retains its ability to induce and to conduct new bone formation, having a major requirement of stability and a healthy host bed. In this regard, as an osteoclastic front advances into the graft, avascular or necrotic, the mesenchymal cells that follow must continuously receive the appropriate signals (osteoinduction) from cytokines, and the graft must be sufficiently stable. Thus, one might consider the necrotic host bone interface within the femoral head as a form of an unstable autograft and that the pathogenesis of osteonecrosis can be considered a mechanically unstable intra-osseous nonunion during the later stages of disease. An intra-osseous nonunion is to be distinctly differentiated from an extra-osseous nonunion wherein fibrous tissue characterizes the ununited bone, whereas the intra-osseous nonunion is characterized by recurring fractures and insufficient internal repair at the necrotic host bone interface. Clearly, if stability of the necrotic segment of autogenous bone can be achieved, either through unloading of the necrotic bone, as suggested by Brown et al., or by providing means for-stabilization so as to facilitate internal repair where osteoinduction remains, union can be expected. The prevention of collapse and the absence of progression will characterize the extent and quality of union, i.e., internal repair.

To date, treatment modalities for osteonecrosis focus on attempts to deliver oxygenated blood to the necrotic bone within the femoral head. In a 1998 January/February article in the Journal of the American Academy of Orthopaedic Surgeons, Urbaniak describes a patent vascular pedicle along a fibula strut within a femoral head 5 days post-operatively. The patency of a typical vascularized fibula graft is usually assumed given the resolution of pain and the lack of progression of disease in a treated patient several years after the index surgery. The formal surgical procedure of decompression of the femoral head with vascularized fibula grafting usually requires prolonged surgery and is a demanding procedure. Vail and Urbaniak reported on donor site morbidity in 247 consecutive grafts in 198 patients at five years follow up. The authors observed an abnormality in 24% of limbs, a sensory deficit in 11.8%, and 2.7% had motor weakness. Other complications reported by Urbaniak and Harvey in 822 vascularized fibula graft procedures include superficial wound infections in two patients, and thromboembolic events in three patients.

Recently, Zimmer began an IDE study using a proprietary material, Hedrocel (trabecular metal, tantalum) as a mechanical device to fill a surgically created void in a femoral neck of a decompressed femoral head. The trabecular metal has a compressive and an elastic modulus similar to cancellous bone. The current IDE study is designed to evaluate the safety and efficacy of trabecular metal in the treatment of patients with early stage disease. The frictional properties of trabecular metal interfaced against cancellous bone are outlined as means for securing the implant within host bone. More importantly, the current investigation is of a nature thought to promote revascularization of the femoral head. Trabecular metal is osteoconductive and promotes bony ingrowth, however, a human allograft will only support “limited internal repair,” therefore, it stands to reason that bony ingrowth into trabecular metal would be quite limited as well. Regarding trabecular metal, bony ingrowth is unidirectional growth, i.e., growth from the surrounding bone into the trabecular metal implant. As an aside, Zimmer promotes an acetabular component in which trabecular metal overlies the outer surface of the component. Bony ingrowth, however limited, is promoted along the surface of the implant as means for establishing its stable fixation. In this setting, bony ingrowth into the implant is ideal. However, trabecular metal or any synthetic component juxtaposed necrotic bone will not promote (induce) new bone formation. Bone ingrowth into trabecular metal is a physical event (osteoconduction), is characterized by a lack of osteoinduction, and is limited (minimal internal repair) as recognized by Enneking. More specifically, an acetabular component with trabecular metal on its outer surface has clinical value, whereas a column of trabecular metal, characterized by limited internal repair, within the femoral neck of a patient with osteonecrosis has less than obvious clinical value. One might surmise that complete débridement of the femoral head of necrotic bone and subsequent stabilization with trabecular metal may very well serve the clinical objectives of operative treatment from a mechanical perspective. However, it is more clinically efficacious to stabilize the femoral head with autogenous cancellous bone. More succinctly, cancellous bone, once incorporated, promotes and supports appositional new bone formation, an absolute requirement to ensure the longevity of the femoral head after decompression/débridement.

With the above understanding as outlined, one might be principally inclined to (1) adequately débride a femoral head of necrotic bone and subsequently assess the adequacy of such débridement with use of an osteodwelling catheter, (2) replace the necrotic bone with viable autologous cancellous bone, and (3) provide means for structural support to a region of overlying cartilage. It is the purpose of the invention described herein to ensure the clinical success of these objectives by using a novel device and a minimally invasive surgical technique to assess the adequacy of débridement of the femoral head.

2. Information Disclosure Statement

Bone grafting is among one of the most frequently performed surgical procedures by surgeons challenged with reconstructing or replacing skeletal defects. Over the years, several techniques have been devised to obtain and implant autologous bone. Scientist and clinicians have sought and defined the essential elements of bone healing and have further desired to secure these elements when considering the benefits of various types of bone grafting techniques. Recently, scientific inquiry has been directed toward understanding the role of bone morphogenic protein (BMP) in the process of new bone formation. What we have learned is that a simple fracture incites a tremendous cascade of events that lead to new bone formation, and that reducing this cascade to a product that can be sold is a difficult task, if not impossible. Nonetheless, orthopaedic surgeons continue to manage complex fractures, which occur daily. Therefore, if one is to appreciate the invention at hand the essentials of fracture healing and new bone formation must be understood.

The essential elements required for bone regeneration are osteoconduction, osteoinduction, and osteogenic cells. In this regard, autogenous bone is the gold standard for bone harvesting. Cancellous bone, as does cortical bone, contains all of these elements but lacks structural integrity. Cortical bone has structural integrity but is limited in quantity. At the histologic and metabolic level, cortical bone is 4 times as dense as cancellous bone, and cancellous bone is 8 times as metabolically active as cortical bone. Further, clinicians have recognized the consequences of donor site morbidity and prolonged hospitalization after a traditional harvesting technique. To circumvent some of these issues, numerous synthetic bone like products have been made available for general use. Each product attempts to exploit one or more of the three essential elements of bone regeneration described above. Although many of these products, e.g., Pro Osteon, INTERPORE, Collagraft, ZIMMER and others are unique, they remain expensive.

To define a less invasive technique for bone harvesting, percutaneous methods have been described. The recently developed techniques simply involve using a coring cylindrical device to obtain a segment of bone. David Billmire, M.D. describes this technique in his article, Use of the CORB Needle Biopsy for the Harvesting of Iliac Crest Bone Graft, PLASTIC AND RECONSTRUCTIVE SURGERY, February 1994. Billmire makes no effort to ensure the quality of the harvested bone but rather describes a power-driven counter-rotating hollow needle as cutting through bone and soft tissue. Michael Saleh describes a percutaneous technique for bone harvesting in his article, Bone-Graft Harvesting: A percutaneous Technique, Journal of Bone and Joint Surgery [Br] 1991; 73-B: 867-8. The author describes using a trephine to twist and lever out a core of bone of 8 mm in size. INNOVASIVE DEVICES describes using their COR™ System for arthroscopic bone graft harvesting. This system describes a disposable cutter having a distal cutting tooth projected into the lumen of the Harvester. This cutting tooth ensures that all harvested osteochondral bone grafts will have a uniform dimension. This cutting tool also serves as means for removing the harvested bone from its donor site. Further, the plunger of the COR™ System is used to disengage gently the harvested bone so as to maintain the overall length of the graft. This concept is absolutely essential to the successful use of the COR™ System as these precisely obtained samples of osteochondral bone are implanted into pre-drilled osteochondral defects within the knee. Further, a vacuum of any sort could not be used on the COR™ System, as the vacuum would simply continue to extract water from the knee joint thereby failing to create an effective pressure drop across the harvested bone and loss of operative visualization. Brannon, in U.S. Pat. No. 6,007,496 describes the use of a vacuum apparatus to create a pressure drop across an osteopiston of bone. Scarborough et al., in U.S. Pat. No. 5,632,747 described a device for cutting short segment dowels from a bone mass. The above prior art does not describe a method for evaluating the adequacy of debridement of a femoral head although the bone graft obtained using any of the prior art devices is traditionally used to graft the femoral head. The novel device describe herein promotes evidence based, medicine, by promoting a treatment regimen that is both quantitative and qualitative. Additional patents include U.S. Pat Nos. 6,165,196 and 6,299,599.

When considering bone for grafting purposes, the recipient site must be considered as well. Failure to achieve bony union at a fracture site or bony fusion at a fusion site may be caused by several factors. Often, the blood flow is inadequate at the fracture site because of local trauma during the inciting event, as might be the case in osteonecrosis of the femoral head. Further, when considering augmentation of the healing process with bone graft, it is imperative that the grafted bone contains all of the essential elements germane to successful osseous regeneration, namely, osteoconductive elements, osteoinductive elements, and osteoprogenitor cells. Most current devices used for bone grafting focus on quantity, the osteoconductive portion of the harvested bone, and less so on quality, the osteoinductive portion of the harvested bone. Recently, bone substitutes have been developed and can be classified according to the following major categories: 1) Osteoconductive synthetics (Pro Osteon 500), 2) Osteoinductive allograft (Grafton), 3) Osteoinductive biosynthetics (OP-1), 4) Osteoinductive autologous bone marrow aspirates, 5) Osteoconductive/Osteoinductive combination synthetics, and 6) Gene therapy. When implanting the above bone graft substitutes, recognizing the usefulness of a collection of, bone growth elements at the fracture site or those generated during the process of open reduction and internal fixation (ORIF) or any other bony procedure, such as posterior spinal instrumentation, has not been achieved through the development of a simple device to promote in situ bone grafting. In this regard, synthetic alternatives to bone grafting can be used as expanders that can be added to autogenous bone and mesenchymal cells harvested in situ at the fracture site or the surgical site. This approach will indeed ensure that all patients are given an optimal opportunity for bony union or bony fusion.

To recognize the issues at hand governing the invention described herein, a simple discussion of biomechanics, physiology, and general physics is warranted and presented in support hereof.

Bone is a viscoelastic material, and as such, it behaves predictably along its stress strain curve when axially loaded in either tension or compression. The key word here is viscoelastic. The prefix “visco” describes the fluid component of the material being tested and the suffix “elastic” describes the recoil potential of the material being tested. The ratio of stress:strain is Young's Modulus. Clearly, a spring is fully elastic. One may place a tension force on a spring, but when the tension is released, the spring recoils to its original length. A syringe, on the other hand, with a thin hypodermic needle attached, is considered viscoelastic. In other words, the amount of deformation observed is time dependent. Simply, the deformation will remain after the tension is removed. Consider one throwing Silly Putty against the ground and observing it bounce versus letting the material sit on a counter for several hours. One should appreciate that minimal deformation occurs when the Silly Putty bounces from the floor versus sitting it on a counter for several hours. The deformation is time dependent because of the internal fluid properties of the material; an amount of time is required to observe a net fluid flow. Bone behaves in a similar fashion, but has the additional property of being able to respond to a given stress by forming new bone. When bone fails to respond favorably, it fractures.

The physiologic properties of bone hinge on the fluid elements that govern bone regeneration, namely, bone morphogenic protein, various hormones, and osteoprogenitor cells. These fluid elements are important to the physiologic function of bone and are found within the bone marrow and the circulatory system. Appreciate that there is a net flow of these elements as bone bares a daily physiologic load during normal walking. Since the circulatory system is a closed system, a net loss of these fluid elements is not observed but rather continuous remodeling of bone and metabolic maintenance of the various cells and proteins as they age and become nonfunctional.

Bone is incompressible above or below its elastic limit, i.e., Young's Modulus. Poisson's ratio is used to describe this behavior and is defined as follows:

v=−(Δd/d₀)/(ΔI/I₀) (1).

Poisson's ratio can be thought of as a measure of how much a material thins when it is stretched, consider taffy, or how much a material bulges when it is compressed. Regarding bone, one does not necessarily observe an increase in volume when it is compressed, but rather an increase in the density as bone remodels along the lines of stress, i.e., form follows function, Wolf's Law. When bone is compressed beyond its elastic limit, it fractures, i.e., it expands, and therefore, its area will increase in a direction perpendicular to the line of force. The fracture observed occurs in the osteoconductive portion of bone, and a fluid flow will occur, as a result of the fracture, within the osteoinductive portion of bone.

The physiology of bone form and function is clear, but what a physician may observe through a series of x-rays may vary from patient to patient. Clearly then what we look for on a x-ray is evidence of healing, and in this regard, fracture healing is divided into at least four categories as follows: 1) inflammatory stage, 2) soft callus stage, 3) hard callus stage, and 4) remodeling stage. Each of these stages has clinical parameters that can be evaluated at the,bedside. It is important to note, however, that any healing process in the human begins with clot formation; consider a simple,laceration. Thus, fracture healing begins with clot formation. However, this stage of fracture healing does not have a clinical parameter unless the fracture is considered an open fracture and the absence of bleeding is observed.

The continuos fluid nature of whole blood (formed elements, i.e., blood cells; serine proteases, i.e., clotting factors; proteins, carbohydrates, electrolytes and hormones) while circulating in the vascular system is substantially maintained by the endothelial lining along the vessel walls. When these circulating serine proteases are exposed to subendothelial collagen or surfaces other than endothelial cells, i.e., abnormal surfaces, platelets aggregate and the clotting cascade is initiated. Blood without formed elements is considered plasma, while plasma without clotting factors is considered serum. A collection of autogenous bone growth elements is considered any and all factors germane to bone formation.

The clotting cascade is divided into two arms; the intrinsic pathway, i.e., local tissue trauma incites clot formation through exposure of the subendothelial collagen to circulating serine proteases and platelets; and the extrinsic pathway which incites clot formation through the activation of Factor VII serine protease and by tissue thromboplastin released from damaged cells. Both pathways then converge on Factor X serine protease. Regarding platelets, these cells are first to arrive and become adherent to injured tissue and form a platelet plug. Adherent platelets are activated platelets and as such release hemostatic agonist and autologous growth factors through a process of degranulation. The hemostatic agonists promote clot formation to ensure that the bleeding stops, while the autologous growth factors initiate the healing process of the injured tissue. Unique to bone is that its healing process is more regenerative of new bone formation as opposed to reparative which is more indicative of scar formation. Scar formation in fracture healing is a nonunion. Further, when bone fractures as a result of surgical or unintentional trauma, a collection of bone growth elements are generated directly within the fracture that contain both fluid and non-fluid components. Within the fluid component are platelets, blood and bone marrow mesenchymal cells, collagen and noncollagenous proteins, and small spicules of bone. The solid component is considered the bony fragments. ORIF is specifically designed to restore length and alignment of the fractured bone through rigid fixation of the non-fluid component. Bone grafting is used when it is determined preoperatively that the structural integrity and the quantity of the bony fragments are insufficient to allow ORIF. Clearly, the collection of bone growth elements required for bony union is present at the fracture site at the time of surgical (core decompression) or unintentional trauma. It stands to reason that in situ autologous bone growth elements, fluid, and non-fluid, should be retained and used in conjunction with means for stabilizing the intra-osseous nonunion within the osteonecrotic femoral head. In situ autologous bone growth factors at a given fracture site unequivocally include the appropriate level of BMP's and other noncollagenous proteins at the various stages of fracture healing as described above. Understanding the physiology of new bone formation, a reparative process, will lend credence to how one should collect and use bone graft elements harvested in situ or from a second operative site.

The above discussion outlines several important areas related to osteonecrosis, specifically, the geometry of the core retrieval process (pending patent, application Ser. No. 09/957,803) and the clotting cascade as it relates to new bone formation. A second pending application Ser. No. (09/957,817) describes the technique and tools necessary for endoscopic visualization of a necrotic segment of bone. It is the intent of this patent to add another component germane to understanding the pathophysiology and treatment of osteonecrosis so as to promote evidenced based medicine. Ficat and Arlet described elevated intraosseous venous pressure in all stages of osteonecrosis and since this time, many authors have ubiquitously concluded that venous hypertension is a major cause of disease. Others have attempted to measure the venous pressure within the femoral head by using washout techniques. One would place a needle into the substance of the femoral neck and measure the entry pressure. With the aid of fluoroscopy, a contrast material is then injected into the femoral neck and the rapidity at which the contrast material dissipates is used as a marker for venous congestion and thus venous hypertension. The invention of this patent application is designed to provide means to asses the arterial pulse pressure within the femoral and femoral neck simultaneously, thereby allowing one to compare pressures therein. Further, one is able to evaluate the quality of the arterial pressure as well as the venous outflow pressure.

### SUMMARY AND OBJECTS OF THE PRESENT INVENTION

It is an object of the present invention to provide means to measure the arterial pulse pressure within an osteonecrotic bone cavity within a femoral head.

It is yet a further object of the present invention to provide means to measure the arterial pulse pressure within a femoral neck.

It is yet a further object of the present invention to provide means to measure the venous outflow pressure within an osteonecrotic bone cavity within the femoral head.

It is yet a further object of the present invention to provide means to measure the venous outflow pressure within a femoral neck.

It is yet a further object of the present invention to provide means continuously anti-coagulating blood inclined to flow into the femoral head and the femoral neck.

It is yet a further object of the present invention to provide real-time clinical data that is useful in promoting evidence based medicine in the treatment of osteonecrosis of the femoral head.

It is yet a further object of the present invention to provide means to partition an osteonecrotic bone cavity from a femoral neck.

### SUMMARY

The present invention describes a novel and unobvious method for determining the quantity and the quality of arterial and venous pressure within the femoral head and the femoral neck. A first expanding-mechanism partitions an osteonecrotic bone cavity from the femoral neck and thereby allows venous and arterial pressure to be measured therein. Contrast material may then be injected so as to evaluate the outflow properties of the osteonecrotic bone cavity. A second serial expanding mechanism is then expanded to establish an osteofluid chamber within the femoral neck. Within the osteofluid chamber, the venous and arterial pressures may be obtained and then compared to the pressures within the femoral head. Contrast material may also be injected into the osteofluid chamber so as to evaluate the venous outflow properties of the femoral neck. A tracing of the pressures can be obtained and recorded intraoperatively to become part of the patient's medical record. The osteodwelling catheter of the present invention is of a size and shape adapted to pass through an endoscope thereby allowing placement of the osteodwelling catheter under direct visualization.

### BRIEF DESCRIPTION OF THE DRAWINGS

FIG. 1 is a sectional anteroposterior view of a femoral head and neck having an osteonecrotic segment bone having been removed therefrom. An osteonecrotic cavity is shown.

FIG. 2 is the osteodwelling catheter of the present invention having a distal catheter end and a proximal arborescent end.

FIG. 3 is a sectional view of the osteodwelling catheter as shown in FIG. 2. In this view, one can visually observe the flow paths and the passageways.

FIG. 4 is a sectional view of the osteodwelling catheter as shown in FIG. 3. In this view, the inflatable balloons are alternately expansively inflated.

FIG. 5 is a close-up view of the osteodwelling catheter having been inserted into an osteocentral canal of a femoral neck. The inflatable balloons have been expanded so as to partition the osteonecrotic bone cavity as shown in FIG. 1. The osteochambers and the osteohermetic chambers are in view as well.

FIG. 6 is a view of the femoral head and neck with the osteoendoscopic cylinder having been inserted therein. The endoscope is shown having been passed through the osteoendoscopic cylinder, wherein the osteodwelling catheter is shown having been passed therethrough. The distal catheter end of the osteodwelling catheter is positioned so as to cause the first lumen to lie juxtainferior to the osteonecrotic bone cavity.

FIG. 7 is a view of the femoral head and neck having the first dynamic anchoring mechanism anchored thereto the distal bony region of the femoral neck. An arrow is shown to emphasize the direction of retraction of the endoscope coaxially about the osteodwelling catheter.

FIG. 8 is yet another view of the femoral head and neck wherein the endoscope has been retracted proximally to visually observe the second deflated visual surface area.

FIG. 9 is yet another view of the femoral head and neck wherein the second inflatable balloon has been alternately expansively inflated so as to establish an osteofluid chamber thereabout the middle catheter region of the osteodwelling catheter. In the configuration as shown in FIG. 9, contrast material may been injected into the femoral head and femoral neck. Alternately, a pressure transducer may be attached to the arborescent end the osteodwelling catheter to determine the intraosseous pressures within the femoral head and neck.

### DESCRIPTION OF THE PREFERRED EMBODIMENTS

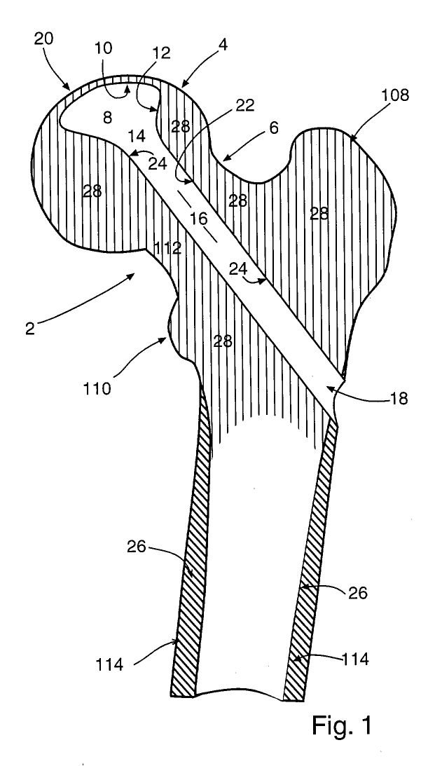

There shown generally at 2 in FIG. 1 is an anteroposterior view of a proximal femur having a femoral head 4 and a femoral neck 6 in anatomic confluency with a greater trochanter 108 and a lesser trochanter 110. Cancellous bone 28 is substantially confluent throughout the femoral head 4 and can be characterized by a plurality osteochambers 30 at the histologic level, the femoral neck 6 and the greater trochanter 108, except for an osteonecrotic bone cavity 8 having been created by instrumentation that is the subject of a co-pending patent (application Ser. No. 1234). The anatomic orientation described for purposes of this invention show that the femoral head 4 is situated distally while the greater and lesser trochanters are situated proximally. The femoral neck 6 establishes a cancellous bony pathway 112 therebetween the femoral head distally and the greater and lesser trochanters proximally. The greater trochanter 108 and the lesser trochanter 110 are in anatomic confluency with a femoral cortical shaft 26. The femoral cortical shaft 26 is comprised of cortical bone 114. In FIG. 1, an osteocentral canal 16 is shown having a longitudinal canal surface 24. A distal bony region 22 is shown juxtainferior to the osteonecrotic bone cavity 8. The osteocentral canal 16 includes a distal neck portal 14 and a proximal neck portal 18. Subchondral bone 10 is seen in this view and is juxtainferior to the cartilaginous surface 20 of the femoral head 4. The osteonecrotic bone cavity includes a sidewall 12 that is comprised of the cancellous bone 28. The femoral head and neck of this view have been prepared for endoscopic visualization and osteodwelling catheter placement.

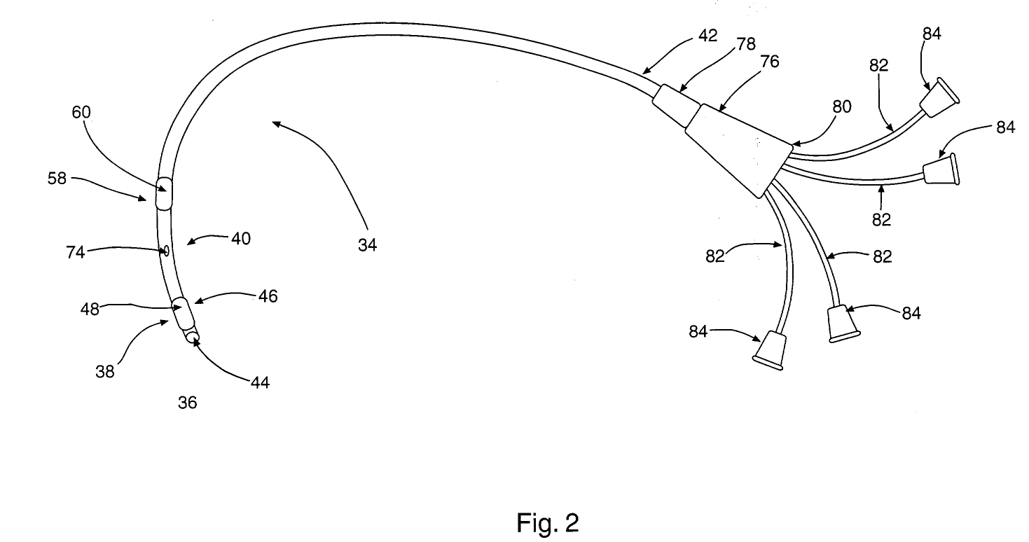

Referring now to FIG. 2, there shown generally at 34 is the osteodwelling catheter of the present invention wherein a distal catheter end 36 is shown. The osteodwelling catheter further includes a first lumen 44 for allowing a fluid to flow therethrough. The osteodwelling catheter further includes a distal catheter region 38. Operationally disposed thereabout the distal catheter region is a first dynamic anchoring mechanism 46. The first dynamic anchoring mechanism 46 comprises a first inflatable balloon 48 having a first deflated visual surface area 50. The first visual surface area 50 is of a size, shape and dimension adapted to be visualized with an endoscope while placed in the femoral head 4. A proximal catheter end 42 is shown and is in confluency with a distal confluent receiving end 78 of a centralizing hub 76. A proximal arborescent end 80 is shown having a plurality of catheters 82 extending proximally therefrom. The plurality of catheters includes a female hub 84 attached to one end thereof.

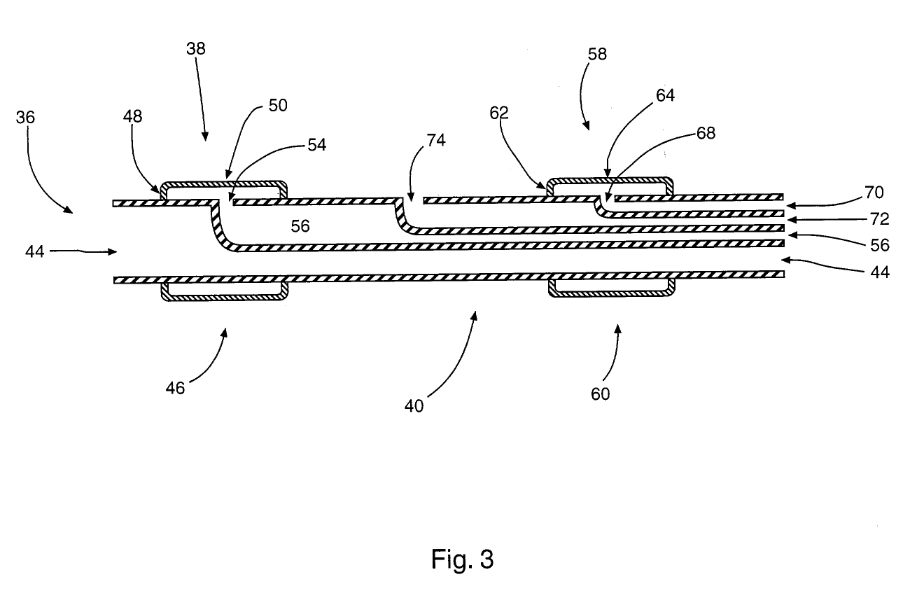

FIG. 3 is an expanded and sectional view of the osteodwelling catheter as shown in FIG. 2. In this sectional view, the first lumen 44 is more readily seen as is the first and second dynamic anchoring mechanisms 46 and 60, respectively. The first dynamic anchoring mechanism 46 thereabout a distal catheter region 38 includes an inflatable balloon 48 having a first deflated visual-surface area 50. In operational continuity is a first aperture 54 having a first passageway 56 extending proximally therefrom. In this view, the middle catheter region 40 is more readily seen and includes a fluid opening 74 having a second lumen 72 extending proximally therefrom. Further in this view is the second dynamic anchoring mechanism 60 thereabout a proximal catheter region 58 comprising a second inflatable balloon 62 having a second deflated visual surface area 64. In operational continuity is a second aperture 68 having a second passageway 70 extending proximally therefrom. One should appreciate that the first and the second fluid paths, as well as the first and second passageways remain separate. One may be inclined to cause each of these thoroughfares to communicate, however, such confluency would have no functional purpose within a segment of bone. Further in this view, one can observe the fluid paths 44 and 72, as well as the passageways 56 and 70. The inflatable balloons are constructed of a material designed to resist laceration and may further be constructed of a material having a corrugated surface, a beaded surface, or an irregular surface so as to increase the frictional resistance at the juncture thereof and the longitudinal canal surface 24. It is an object of the present invention to cause the inflatable balloons to alternately and expansively inflate thereby increasing the frictional resistance between the inflated balloon and the longitudinal canal surface.

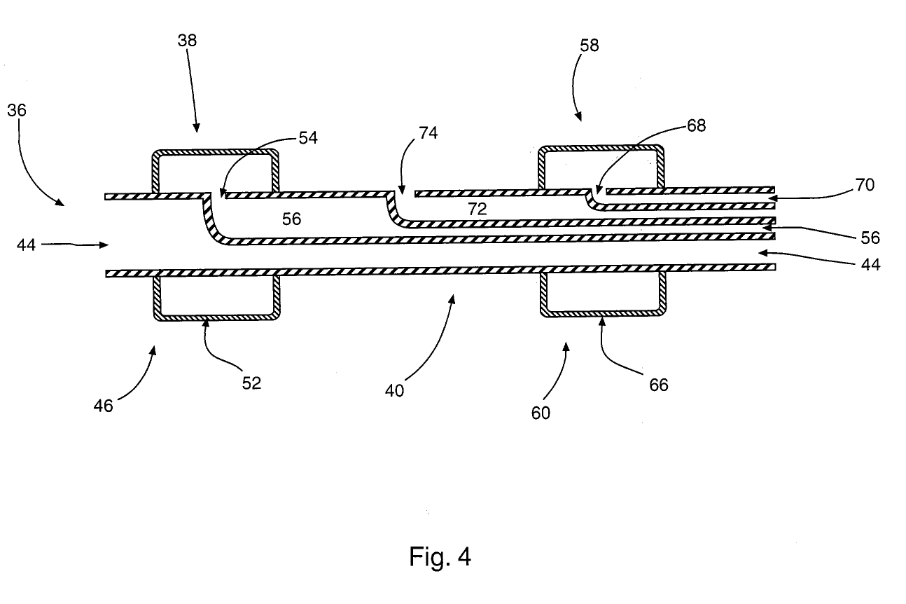

Now turning to FIG. 4, there shown in sectional view is the osteodwelling catheter of the present invention wherein the inflatable balloons have been inflated. More specifically, a first alternately expansively inflated bony contact surface area 52 having a first aperture in operational continuity therewith is readily seen. Here again, the middle catheter region 40 is shown having a fluid opening 74. Further in this view, a second alternately expansively inflated bony contact surface area 66 having a second aperture 68 in operational continuity therewith can be seen. The alternately expansively inflated bony contact surface areas proffer a first and a second surface area to which platelets may aggregate and degranulate and thereby inducing hemostasis locally thereabout the inflated balloons. The local hemostasis is of a magnitude to hermetically partition the osteonecrotic bone cavity from the femoral neck and sequentially establish an osetofluid chamber 106 within the femoral neck.

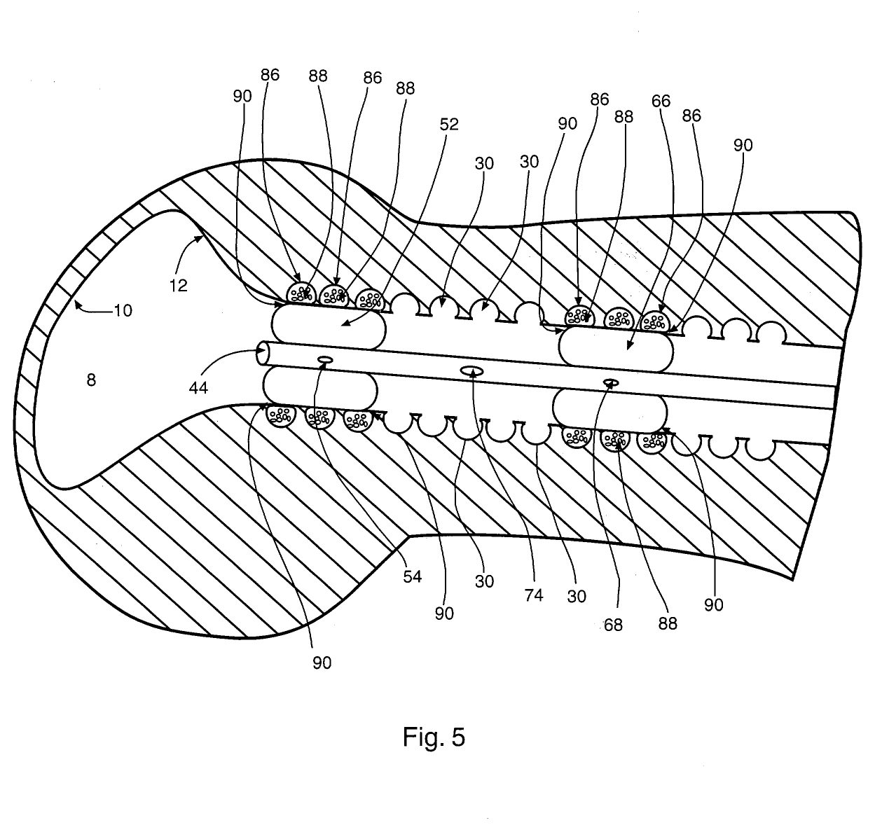

FIG. 5 is an expanded view of the osteocentral canal within the femoral neck. In this view, the osteodwelling catheter is shown having been engaged by the longitudinal canal surface 24 in the region of the distal neck portal 14. In this view, the plurality of osteochambers 30 is shown throughout the osteocentral canal 16. More specifically, a plurality of osteohermetic chambers 86 are shown having been established at the juncture of the first and the second alternately expansively inflated bony contact surface areas 52 and 66, respectively, and the longitudinal canal surface. A collection of platelets 88 can been seen within the plurality of osteohermetic chambers. In this view, the platelets are shown to have induced the formation of clot 90 locally thereabout the first and second dynamic anchoring mechanisms 46 and 60, respectively. The first lumen 44 and the fluid opening 74 can be readily seen.

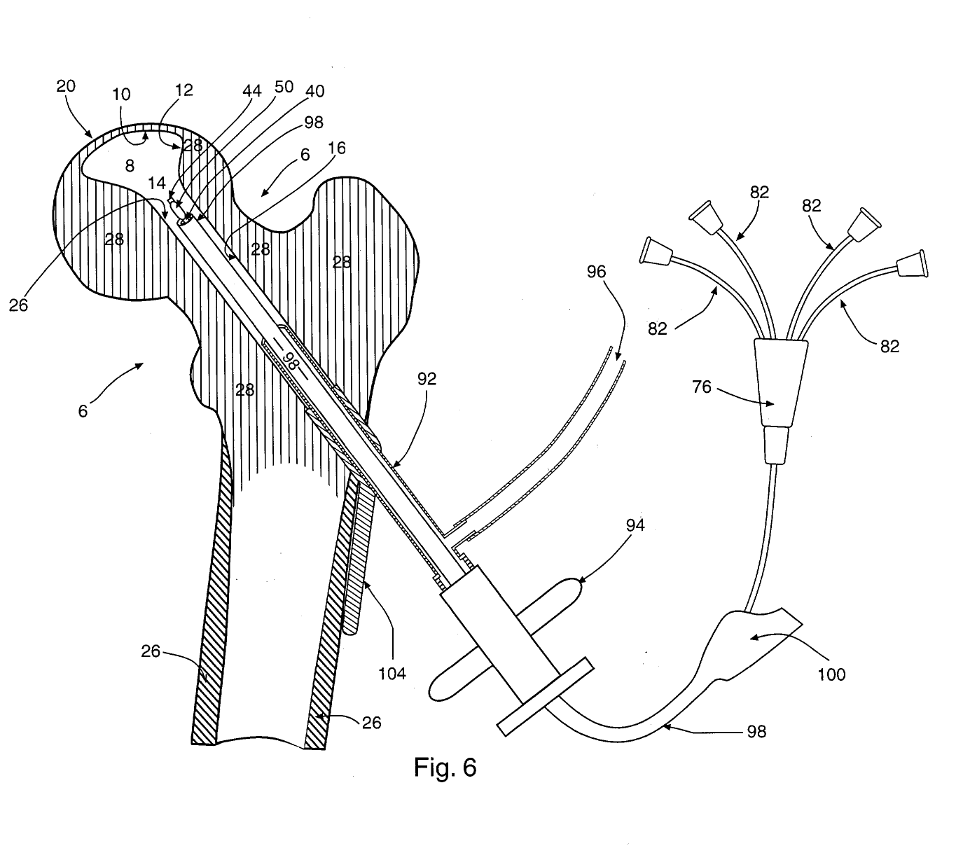

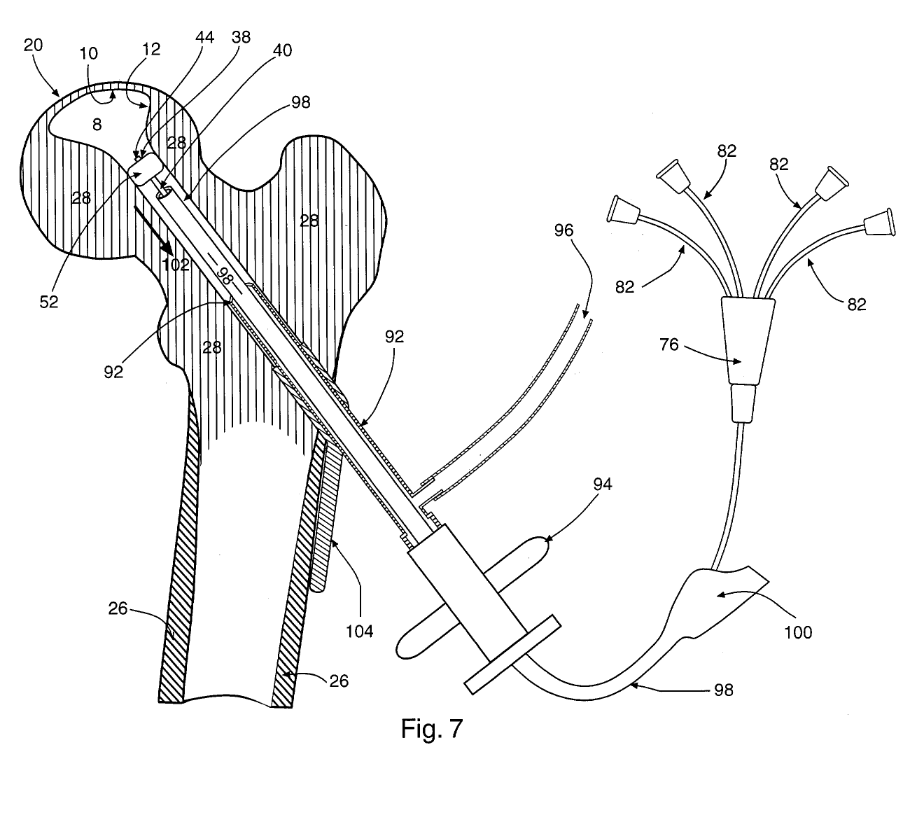

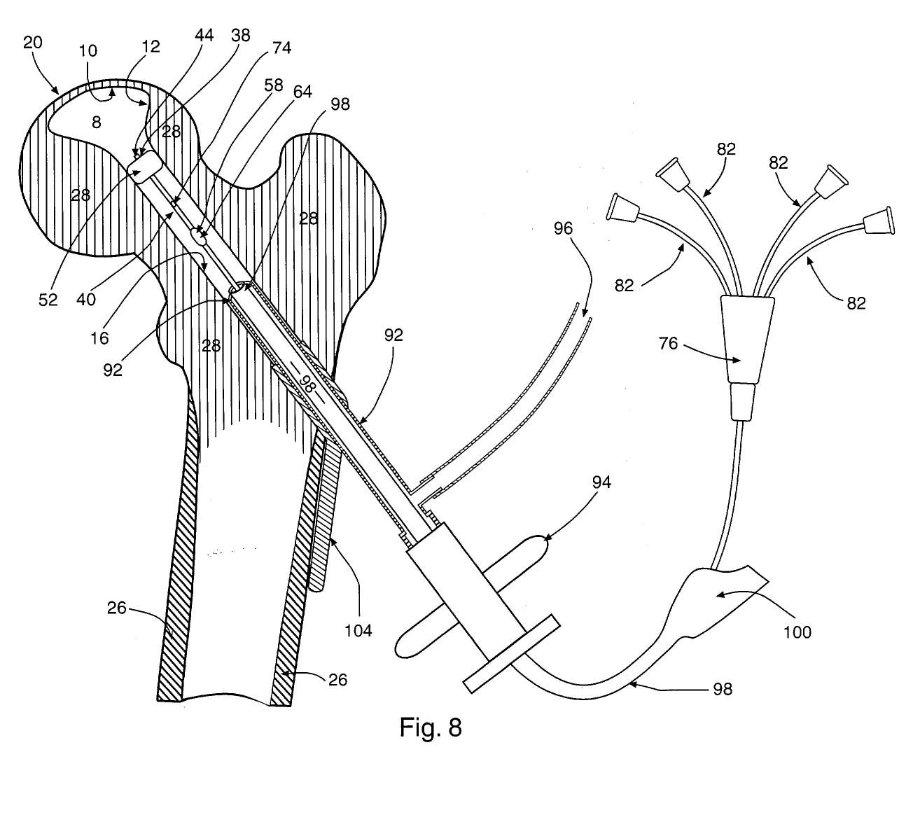

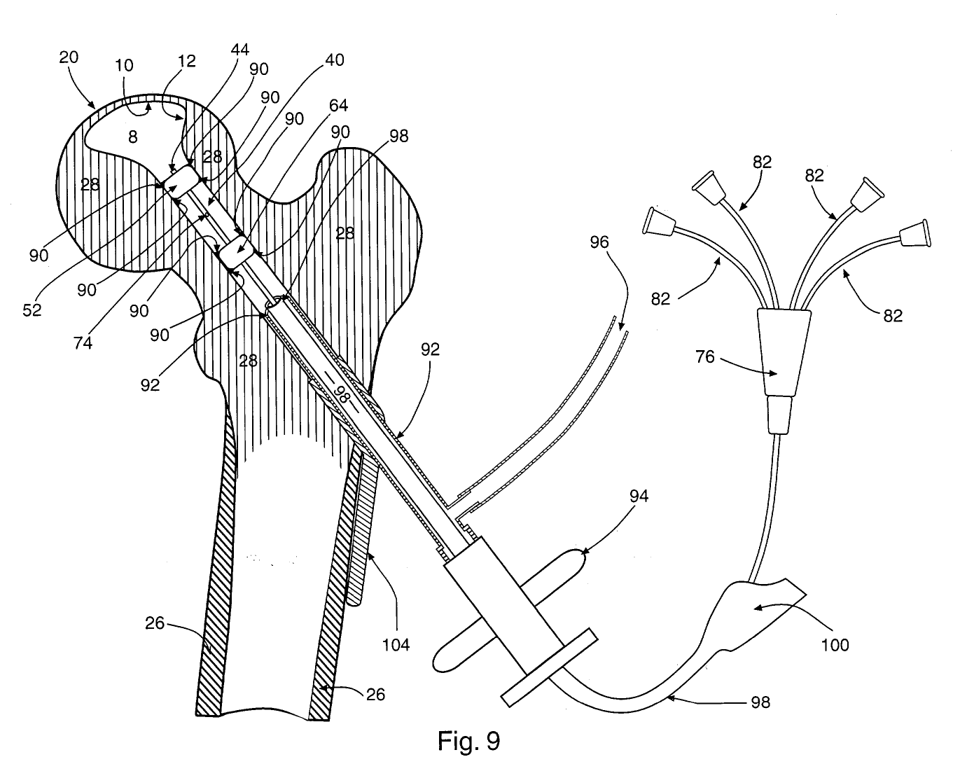

Turning now to FIG. 6 there shown is the osteoendoscopic cylinder 92 having been inserted into the osteocentral canal 16. The osteoendoscopic cylinder is shown having a suction tube 96 and a handle 94 attached thereto. The endoscope 98 is shown having been passed into the femoral neck 6 up to the distal neck portal 14. The osteodwelling catheter is shown having been passed through the endoscope 98 so as to cause the first deflated visual surface area 50 to be visualized. The proximal arborescent end 80 of the centralizing hub 76 is shown. An endoscopic portal plate 104 is shown having engaged the femoral cortical shaft 26. In this configuration, the first inflatable balloon 48 is inflated as shown in FIG. 7 so as to anchor the distal catheter region of the osteodwelling catheter in the region of the distal portal neck 14 and to proffer a first surface area to which platelets can aggregate and degranulate and thereby induce the formation of clot thereabout the first inflatable balloon and hermetically partition the osteonecrotic bone cavity 8 from the osteocentral canal 16. The anchoring is of a magnitude to allow coaxial retraction of the endoscope thereabout the osteodwelling catheter in the direction of the directional arrow 102. The first lumen 44 freely communicates with the osteonecrotic bone cavity 8 so as to allow a contrast material to be delivered therein, or alternately allows one to obtain an arterial or venous pressure. FIG. 8 is the same view as in FIG. 7, however, the endoscope is now shown in a retracted position such that the second deflated visual surface area 64 is positioned to be visualized by the endoscope 98. FIG. 9 is the same view as in FIG. 8, however, in this configuration, the second inflatable balloon 48 is inflated so as to anchor the proximal catheter region of the osteodwelling catheter to the osteocentral canal and to proffer a second surface area to which platelets can aggregate and degranulate and thereby induce the formation of clot 90 thereabout the second inflatable balloon. Having the first and second balloons inflated, an osteofluid chamber 106 is hermetically established thereabout the middle catheter region 40. The fluid opening 74 freely communicates with the osteofluid chamber through which a contrast material can be delivered, or alternately allow one to obtain an arterial or venous pressure. Partitioning the bone and obtaining contrast studies as well as pressure measurements (the pressure within the osteonecrotic bone cavity can be compared to the pressure within the ipsilateral femoral neck and one can further comparatively evaluate the pulse pressures using a pressure transducer) provides needed information in the management of osteonecrosis of the femoral head. Such information will enhance an evidenced based approach to treating this dreaded disease.

What has been described is illustrative only and by no means is intended to represent all embodiments or modifications, as one might conceive an alternative embodiment, however, such alternative embodiment would not and could not deviate from the spirit of the invention. This novel invention is unique and will provide practical clinical information that is obtained non-invasively. More importantly, the invention described herein avoids the need to create increasingly larger cores within the femoral neck. Further, the invention described herein is simple, yet the information obtained with the use of the osteodwelling catheter is useful in the care of the patient with osteonecrosis.

Specification List

2. A proximal femur

4. A femoral head

6. A femoral neck

8. An osteonecrotic bone cavity

10. Subchondral bone

12. A sidewall

14. A distal portal neck

16. An osteocentral canal

18. A proximal neck portal

20. A cartilaginous surface

22. A distal bony region

24. A longitudinal canal surface

26. A femoral cortical shaft

28. Cancellous bone

30. A plurality of osteochambers

32. A proximal bony region

34. An osteodwelling catheter

36. A distal catheter end

38. A distal catheter region

40. A middle catheter region

42. A proximal catheter end

44. A first lumen

46. A first dynamic anchoring mechanism

48. A first inflatable balloon

50. A first deflated visual surface area

52. A first alternately expansively inflated bony contact area

54. A first aperture

56. A first passageway

58. A proximal catheter region

60. A second dynamic anchoring mechanism

62. A second inflatable balloon

64. A second deflated visual surface area

66. A second alternately expansively inflated bony contact area

68. A second aperture

70. A second passageway

72. A second lumen

74. A fluid opening

76. A centralizing hub

78. A distal confluent receiving end

80. A proximal arborescent end

82. A plurality of catheters

84. A female hub

86. A plurality of osteohermetic chambers

88. A collections of platelets

90. Clot

92. An osteoendoscopic cylinder

94. A handle

96. A suction tube

98. An endoscope

100. An endoscopic handle

102. A directional arrow

104. An endoscopic portal plate

106. An osteofluid chamber

108. A greater trochanter

110. A lesser trochanter

112. A bony pathway

114. Cortical bone

Thus having described the invention, what I desire to claim and secure by Letters Patent is:

1. A device for partitioning an osteonecrotic cavity within a femoral head comprising:
an osteodwelling catheter having a distal catheter end, a distal catheter region, a middle catheter region, and a proximal catheter end, said osteodwelling catheter further includes a first lumen extending therethrough so as to establish a first flow path therebetween said distal catheter end and said proximal catheter end, said distal catheter region being interposed between said distal catheter end and said middle catheter region, said distal catheter region includes a first dynamic anchoring mechanism, said first dynamic anchoring mechanism comprising a first inflatable balloon having a first deflated visual surface area and a first alternately expansively inflated bony contact surface area, said first inflatable balloon is functionally disposed thereabout said distal catheter region, said first dynamic anchoring mechanism includes a first aperture in operational continuity with said first inflatable balloon, said first aperture further includes a first passageway extending proximally therefrom and throughout said osteodwelling catheter to said proximal catheter end, said first alternately expansively inflated bony contact surface area is of a size and shape adapted to induce platelets to aggregate at the juncture thereof and a longitudinal canal surface of an osteocentral canal within a femoral neck, the platelet aggregation is of a magnitude to induce hemostasis thereabout said first alternately expansively inflated bony contact surface area so as to hermetically partition the osteonecrotic cavity from the osteocentral canal, said first alternately expansively inflated bony contact surface area is further of a size and shape adapted to substantially induce engagement of said first dynamic anchoring mechanism along the longitudinal canal surface, said engagement is of a magnitude to substantially resist disengagement of said first dynamic anchoring mechanism, said first inflatable balloon being comprised of a material having a property adapted to substantially resist laceration thereof when engaged thereupon the longitudinal canal surface.

2. A device as defined in claim 1 wherein said osteodwelling catheter further includes a proximal catheter region functionally interposed therebetween said middle catheter region and said proximal catheter end, said proximal catheter region includes a second dynamic anchoring mechanism, said second dynamic anchoring mechanism comprising a second inflatable balloon having a second deflated visual surface area and a second alternately expansively inflated bony contact surface area, said second inflatable balloon is functionally disposed thereabout said proximal catheter region, said second dynamic anchoring mechanism includes a second aperture in operational continuity with said second inflatable balloon, said second aperture further includes a second passageway extending proximally therefrom and throughout said osteodwelling catheter to said proximal catheter end, said second alternately expansively inflated bony contact surface area is of a size and shape adapted to substantially induce engagement of said second dynamic anchoring mechanism along the longitudinal canal surface, said engagement is of a magnitude to substantially resist disengagement of said second dynamic anchoring mechanism of said osteodwelling catheter, said second alternately expansively inflated bony contact surface area is further of a size and shape adapted to induce platelets to aggregate at the juncture thereof and the longitudinal canal surface, the platelet aggregation is of a magnitude to substantially induce hemostasis thereabout said second alternately expansively inflated bony contact surface area so as to hermetically establish an osteofluid chamber thereabout said middle catheter region within the femoral neck therebetween said first and said second inflatable balloons, said second inflatable balloon being comprised of a material having a property adapted to substantially resist laceration thereof when engaged thereupon the longitudinal canal surface.

3. A device as defined in claim 1 wherein said osteodwelling catheter further includes a second lumen having a fluid opening functionally disposed thereabout said middle catheter region, said second lumen extending proximally from said fluid opening to said proximal catheter end so as to establish a second flow path therebetween said middle catheter region and said proximal catheter end.

4. A device as defined in claim 1 wherein said proximal catheter end comprises a centralizing hub having a distal confluent receiving end and a proximal arborescent end, said centralizing hub includes means for ensuring said first lumen, said first passageway, said second lumen, and said second passageway remain separate therein, said arborescent end further includes means for securing a plurality of catheters thereto, said plurality of catheters having a female hub at one end thereof.

5. A device for partitioning an osteonecrotic cavity within a femoral head comprising:
an osteodwelling catheter adapted to pass through an endoscope includes a distal catheter end, a distal catheter region, a middle catheter region, and a proximal catheter end, said osteodwelling catheter further includes a first lumen extending therethrough so as to establish a first flow path therebetween said distal catheter end and said proximal catheter end, said distal catheter region being interposed between said distal catheter end and said middle catheter region, said distal catheter region includes a first dynamic anchoring mechanism, said first dynamic anchoring mechanism comprising a first inflatable balloon having a first deflated visual surface area adapted to be visualized by said endoscope, said first dynamic anchoring mechanism further includes a first alternately expansively inflated bony contact surface area, said first inflatable balloon is functionally disposed thereabout said distal catheter region, said first dynamic anchoring mechanism includes a first aperture in operational continuity with said first inflatable balloon, said first aperture further includes a first passageway extending proximally therefrom and throughout said osteodwelling catheter to said proximal catheter end, said first alternately expansively inflated bony contact surface area is of a size and shape adapted to induce platelets to aggregate at the juncture thereof and a longitudinal canal surface of an osteocentral canal within a femoral neck, the platelet aggregation is of a magnitude to induce hemostasis thereabout said first alternately expansively inflated bony contact surface area so as to hermetically partition the osteonecrotic cavity from the osteocentral canal, said first alternately expansively inflated bony contact surface area is further of a size and shape adapted to substantially induce engagement of said first dynamic anchoring mechanism along the longitudinal canal surface, said engagement is of a magnitude to substantially allow telescoping of said endoscope thereabout said osteodwelling catheter, said first inflatable balloon being comprised of a material having a property adapted to substantially resist laceration thereof when engaged thereupon the longitudinal canal surface.

6. A device as defined in claim 5 wherein said osteodwelling catheter further includes a proximal catheter region functionally interposed therebetween said middle catheter region and said proximal catheter end, said proximal catheter region includes a second dynamic anchoring mechanism, said second dynamic anchoring mechanism comprising a second inflatable balloon having a second deflated visual surface area and second alternately expansively inflated bony contact surface area, said second inflatable balloon is functionally disposed thereabout said proximal catheter region, said second dynamic anchoring mechanism includes a second aperture in operational continuity with said second inflatable balloon, said second aperture further includes a second passageway extending proximally therefrom and throughout said osteodwelling catheter to said proximal catheter end, said second alternately expansively inflated bony contact surface area is of a size and shape adapted to substantially induce engagement of said second dynamic anchoring mechanism along the longitudinal canal surface, said engagement is of a magnitude to substantially resist motion of said osteodwelling catheter, said second alternately expansively inflated bony contact surface area is further of a size and shape adapted to induce platelets to aggregate at the juncture thereof and the longitudinal canal surface, the platelet aggregation is of a magnitude to substantially induce hemostasis thereabout said second alternately expansively inflated bony contact surface area so as to hermetically establish an osteofluid chamber thereabout said middle catheter region within the femoral neck therebetween said first and said second inflatable balloons, said second inflatable balloon being comprised of a material having a property adapted to substantially resist laceration thereof when engaged thereupon the longitudinal canal surface.

7. A device as defined in claim 5 wherein said osteodwelling catheter further includes a second lumen having a fluid opening functionally disposed thereabout said middle catheter, region, said second lumen extending proximally from said fluid opening to said proximal catheter end so as to establish a second flow, path therebetween said middle catheter region and said proximal catheter end.

8. A device as defined in claim 5 wherein said proximal catheter end comprises a centralizing hub having a distal confluent receiving end and a proximal arborescent end, said centralizing hub includes means for ensuring said first lumen, said first passageway, said second lumen, and said second passageway remain separate therein, said arborescent end further includes means for securing a plurality of catheters thereto, said plurality of catheters having a female hub at one end thereof.

9. A device for partitioning an osteonecrotic cavity within a femoral head comprising:
an osteodwelling catheter having a distal catheter end, a distal catheter region, a middle catheter region, and a proximal catheter end, said osteodwelling catheter is adapted to pass through an endoscope, said osteodwelling catheter further includes a first lumen extending therethrough so as to establish a first flow path therebetween said distal catheter end and said proximal catheter end, said distal catheter region being interposed between said distal catheter end and said middle catheter region, said distal catheter region includes a first dynamic anchoring mechanism, said first dynamic anchoring mechanism comprising a first expandable mechanism having a first unexpanded visual surface area and a first alternately expanded bony contact surface area, said first expandable mechanism is functionally disposed thereabout said distal catheter region, said first dynamic anchoring mechanism includes a first aperture in operational continuity with said first expandable mechanism, said first aperture further includes a first passageway extending proximally therefrom and throughout said osteodwelling catheter to said proximal catheter end, said first alternately expanded bony contact surface area is of a size and shape adapted to induce platelets to aggregate at the juncture thereof and a longitudinal canal surface of an osteocentral canal within a femoral neck, the platelet aggregation is of a magnitude to induce hemostasis thereabout said first alternately expanded bony contact surface area so as to partition the osteonecrotic cavity from the osteocentral canal, said first alternately expanded bony contact surface area is further of a size and shape adapted to substantially induce engagement of said first dynamic anchoring mechanism along the longitudinal canal surface, said engagement is of a magnitude to substantially resist disengagement of said first dynamic anchoring mechanism and thereby substantially allow telescoping of said endoscope thereabout said osteodwelling catheter.

10. A device as defined in claim 9 wherein said osteodwelling catheter further includes a proximal catheter region functionally interposed therebetween said middle catheter region and said proximal catheter end, said proximal catheter region includes a second dynamic anchoring mechanism, said second dynamic anchoring mechanism comprising a second expandable mechanism having a second unexpanded visual surface area and a second alternately expanded bony contact surface area, said second expandable mechanism is functionally disposed thereabout said proximal catheter region, said second dynamic anchoring mechanism includes a second aperture in operational continuity with said second expandable mechanism, said second aperture further includes a second passageway extending proximally therefrom and throughout said osteodwelling catheter to said proximal catheter end, said second alternately expanded bony contact surface area is of a size and shape adapted to substantially induce engagement of said second dynamic anchoring mechanism along the longitudinal canal surface, said engagement is of a magnitude to substantially resist motion of said osteodwelling catheter, said second alternately expanded bony contact surface area is further of a size and shape adapted to induce platelets to aggregate at the juncture thereof and the longitudinal canal surface, the platelet aggregation is of a magnitude to substantially induce hemostasis thereabout said second alternately expanded bony contact surface area so as to establish an osteofluid chamber thereabout said middle catheter region within the femoral neck therebetween said first and said second expandable mechanisms.

11. A device as defined in claim 9 wherein said osteodwelling catheter further includes a second lumen having a fluid opening functionally disposed thereabout said middle catheter region, said second lumen extending proximally from said fluid opening to said proximal catheter end so as to establish a second flow path therebetween said middle catheter region and said proximal catheter end.

12. A device as defined in claim 9 wherein said proximal catheter end comprises a centralizing hub having a distal confluent receiving end and a proximal arborescent end, said centralizing hub includes means for ensuring said first lumen, said first passageway, said second lumen, and said second passageway remain separate therein, said arborescent end further includes means for securing a plurality of catheters thereto, said plurality of catheters having a female hub at one end thereof.

13. A device for partitioning an osteonecrotic cavity within a femoral head comprising:
an osteodwelling catheter having a distal catheter end, a distal catheter region, a middle catheter region, and a proximal catheter end, said osteodwelling catheter further includes a first lumen extending therethrough so as to establish a first flow path therebetween said distal catheter end and said proximal catheter end, said distal catheter region being interposed between said distal catheter end and said middle catheter region, said distal catheter region includes a first dynamic anchoring mechanism, said first dynamic anchoring mechanism comprising a first inflatable balloon having a first deflated visual surface area and a first alternately expansively inflated bony contact surface area, said first inflatable balloon is functionally disposed thereabout said distal catheter region, said first dynamic anchoring mechanism includes a first aperture in operational continuity with said first inflatable balloon, said first aperture further includes a first passageway extending proximally therefrom and throughout said osteodwelling catheter to said proximal catheter end, said first alternately expansively inflated bony contact surface area is of a size and shape so as to proffer an expanded surface area to which a collection of platelets are encouraged to aggregate, the platelet aggregation is of a magnitude to locally induce hemostasis thereabout said first alternately expansively inflated bony contact surface area so as to hermetically partition the osteonecrotic cavity from an osteocentral canal having a longitudinal canal surface, said first alternately expansively inflated bony contact surface area is further of a size and shape adapted to substantially induce engagement of~said first dynamic anchoring mechanism along the longitudinal canal surface, said engagement is of a magnitude to substantially resist disengagement of said first dynamic anchoring mechanism.

14. A device as defined in claim 13 wherein said first inflatable balloon is comprised of a material having a property adapted to substantially resist laceration thereof when engaged thereupon the longitudinal canal surface.

15. A device as defined in claim 13 wherein said osteodwelling catheter further includes a proximal catheter region functionally interposed therebetween said middle catheter region and said proximal catheter end, said proximal catheter region includes a second dynamic anchoring mechanism, said second dynamic anchoring mechanism comprising a second inflatable balloon having a second deflated visual surface area and a second alternately expansively inflated bony contact surface area, said second inflatable balloon is functionally disposed thereabout said proximal catheter region, said second dynamic anchoring mechanism includes a second aperture in operational continuity with said second inflatable balloon, said second aperture further includes a second passageway extending proximally therefrom and throughout said osteodwelling catheter to said proximal catheter end, said second alternately expansively inflated bony contact surface area is of a size and shape adapted to substantially induce engagement of said second dynamic anchoring mechanism along the longitudinal canal surface, said engagement is of a magnitude to substantially resist disengagement of said second dynamic anchoring mechanism of said osteodwelling catheter, said second alternately expansively inflated bony contact surface area is further of a size and shape adapted to induce platelets to aggregate at the juncture thereof and the longitudinal canal surface, the platelet aggregation is of a magnitude to substantially induce hemostasis thereabout said second alternately expansively inflated bony contact surface area so as to hermetically establish an osteofluid chamber thereabout said middle catheter region within the femoral neck therebetween said first and said second inflatable balloons, said second inflatable balloon being comprised of a material having a property adapted to substantially resist laceration thereof when engaged thereupon the longitudinal canal surface.

16. A device as defined in claim 13 wherein said osteodwelling catheter further includes a second lumen having a fluid opening functionally disposed thereabout said middle catheter region, said second lumen extending proximally from said fluid opening to said proximal catheter end so as to establish a second flow path therebetween said middle catheter region and said proximal catheter end.

17. A device as defined in claim 13 wherein said proximal catheter end comprises a centralizing hub having a distal receiving end and a proximal arborescent end, said centralizing hub includes means for ensuring said first lumen, said first passageway, said second lumen, and said second passageway remain separate therein, said arborescent end further includes means for securing a plurality of catheters thereto, said plurality of catheters having a female hub at one end thereof.

18. A device for establishing a first plurality of osteohermetic chambers within the substance of a bone a comprising:
an osteodwelling catheter having a distal catheter end, a distal catheter region, a middle catheter region, and a proximal catheter end, said osteodwelling catheter further includes a first lumen extending therethrough so as to establish a first flow path therebetween said distal catheter end and said proximal catheter end, said distal catheter region being interposed between said distal catheter end and said middle catheter region, said distal catheter region includes a first dynamic anchoring mechanism, said first dynamic anchoring mechanism comprising a first inflatable balloon having a first deflated visual surface area and a first alternately expansively inflated bony contact surface area, said first inflatable balloon is functionally disposed thereabout said distal catheter region, said first dynamic anchoring mechanism includes a first aperture in operational continuity with said first inflatable balloon, said first aperture further includes a first passageway extending proximally therefrom and throughout said osteodwelling catheter to said proximal catheter end, said first alternately expansively inflated bony contact surface area is of a size and shape adapted to establish and to localize said first plurality of osteohermetic chambers thereabout said first alternately expansively inflated bony contact surface area within an osteocentral canal having a longitudinal canal surface, the first plurality of osteohermetic chambers are of a dimension adapted to induce platelets to aggregate and to degranulate therein so as to locally promote hemostasis thereabout said first alternately expansively inflated bony contact surface area, the hemostasis is of a degree to partition the bone.

19. A device and defined in claim 18 wherein said first alternately expansively inflated bony contact surface area is further of a size and shape adapted to substantially induce mechanical engagement of said first dynamic anchoring mechanism along the longitudinal canal surface, said mechanical engagement is of a magnitude to substantially allow coaxial retraction of an endoscope thereabout said osteodwelling catheter.

20. A device as defined in (claim 18 wherein said first inflatable balloon is comprised of a material having a property adapted to substantially resist laceration thereof when engaged thereupon the longitudinal canal surface.

21. A device as defined in claim 18 wherein said osteodwelling catheter further includes a proximal catheter region functionally interposed therebetween said middle catheter region and said proximal catheter end, said proximal catheter region includes a second dynamic anchoring mechanism, said second dynamic anchoring mechanism comprising a second inflatable balloon having a second deflated visual surface area and a second alternately expansively inflated bony contact surface area, said second inflatable balloon is functionally disposed thereabout said proximal catheter region, said second dynamic anchoring mechanism includes a second aperture in operational continuity with said second inflatable balloon, said second aperture further includes a second passageway extending proximally therefrom and throughout said osteodwelling catheter to said proximal catheter end, said second alternately expansively inflated bony contact surface area is of a size and shape adapted to substantially establish and to substantially localize a second plurality of osteohermetic chambers thereabout said second alternately expansively inflated bony contact surface area within the osteocentral canal, the second plurality of osteohermetic chambers are of a dimension adapted to induce platelets to aggregate and to degranulate therein so as to locally promote hemostasis thereabout said second alternately expansively inflated bony contact surface area, the hemostasis is of a degree to partition the bone so as to hermetically establish an osteofluid chamber thereabout said middle catheter region therebetween said first and said second inflatable balloons, said second alternately expansively inflated bony contact surface area is further of a size and shape adapted to substantially induce mechanical engagement of said second dynamic anchoring mechanism along the longitudinal canal surface, said mechanical engagement is of a magnitude to substantially resist motion of said osteodwelling catheter, said second inflatable balloon being comprised of a material having a property adapted to substantially resist laceration thereof when engaged thereupon the longitudinal canal surface.

22. A device as defined in claim 18 wherein said osteodwelling catheter further includes a second lumen having a fluid opening functionally disposed thereabout said middle catheter region, said second lumen extending proximally from said fluid opening to said proximal catheter end so as to establish a second flow path therebetween said middle catheter region and said proximal catheter end.

23. A device as defined in claim 18 wherein said proximal catheter end comprises a centralizing hub having a distal confluent receiving end and a proximal arborescent end, said centralizing hub includes means for ensuring said first lumen, said first passageway, said second lumen, and said second passageway remain separate therein, said arborescent end further includes means for securing a plurality of catheters thereto, said plurality of catheters having a female hub at one end thereof.

24. A device for establishing a first plurality of osteohermetic chambers within the substance of a bone a comprising:
an osteodwelling catheter having a distal catheter end, a distal catheter region, a middle catheter region, and a proximal catheter end, said osteodwelling catheter further includes a first lumen extending therethrough so as to establish a first flow path therebetween said distal catheter end and said proximal catheter end, said distal catheter region being interposed between said distal catheter end and said middle catheter region, said distal catheter region includes a first dynamic anchoring mechanism, said first dynamic anchoring mechanism comprising a first inflatable balloon having a first deflated visual surface area and a first alternately expansively inflated bony contact surface area, said first inflatable balloon is functionally disposed thereabout said distal catheter region, said first alternately expansively inflated bony contact surface area is of a size and shape adapted to establish and to localize said first plurality of osteohermetic chambers thereabout said first alternately expansively inflated bony contact surface area within an osteocentral canal having a longitudinal canal surface, the first plurality of osteohermetic chambers are of a dimension adapted to induce platelets to aggregate and to degranulate therein so as to, locally promote hemostasis thereabout said first alternately expansively inflated bony contact surface area, the hemostasis is of a degree to partition the bone.

25. A device as defined in claim 24 wherein said first dynamic anchoring mechanism includes a first aperture in operational continuity with said first inflatable balloon, said first aperture includes a first passageway extending proximally therefrom and throughout said osteodwelling catheter to said proximal catheter end.

26. A device and defined in claim 24 wherein said first alternately expansively inflated bony contact surface area is further of a size and shape adapted to substantially induce mechanical engagement of said first dynamic anchoring mechanism along the longitudinal canal surface, said mechanical engagement is of a magnitude to substantially allow coaxial retraction of an endoscope thereabout said osteodwelling catheter.

27. A device as defined in claim 24 wherein said first inflatable balloon is comprised of a material having a property adapted to substantially resist laceration thereof when engaged thereupon the longitudinal canal surface.

28. A device as defined in claim 24 wherein said osteodwelling catheter further includes a proximal catheter region functionally interposed therebetween said middle catheter region and said proximal catheter end, said proximal catheter region includes a second dynamic anchoring mechanism, said second dynamic anchoring mechanism comprising a second inflatable balloon having a second deflated visual surface area and a second alternately expansively inflated bony contact surface area, said second inflatable balloon is functionally disposed thereabout said proximal catheter region, said second dynamic anchoring mechanism includes a second aperture in operational continuity with said second inflatable balloon, said second aperture further includes a second passageway extending proximally therefrom and throughout said osteodwelling catheter to said proximal catheter end, said second alternately expansively inflated bony contact surface area is of a size and shape adapted to substantially establish and to substantially localize a second plurality of osteohermetic chambers thereabout said second alternately expansively inflated bony contact surface area within the osteocentral canal, the second plurality of osteohermetic chambers are of a dimension adapted to induce platelets to aggregate and to degranulate therein so as to locally promote hemostasis thereabout said second alternately expansively inflated bony contact surface area, the hemostasis is of a degree to partition the bone so as to hermetically establish an osteofluid chamber thereabout said middle catheter region therebetween said first and said second inflatable balloons, said second alternately expansively inflated bony contact surface area is further of a size and shape adapted to substantially induce mechanical engagement of said second dynamic anchoring mechanism along the longitudinal canal surface, said mechanical engagement is of a magnitude to substantially resist motion of said osteodwelling catheter, said second inflatable balloon being comprised of a material having a property adapted to substantially resist laceration thereof when engaged thereupon the longitudinal canal surface.

29. A device as defined in claim 24 wherein said osteodwelling catheter further includes a second lumen having a fluid opening functionally disposed thereabout said middle catheter region, said second lumen extending proximally from said fluid opening to said proximal catheter end so as to establish a second flow path therebetween said middle catheter region and said proximal catheter end.

30. A device as defined in claim 24 wherein said proximal catheter end comprises a centralizing hub having a distal confluent receiving end and a proximal arborescent end, said centralizing hub includes means for ensuring said first lumen, said first passageway, said second lumen, and said second passageway remain separate therein, said arborescent end further, includes means for securing a plurality of catheters thereto, said plurality of catheters having a female hub at one end thereof.
